(19) United States
(12) Patent Application Publication
(10) Pub. No.: US 2010/0305514 A1
(43) Pub. Date: Dec. 2, 2010

(54) INTRAVITREAL INJECTION SYSTEM HAVING COAXIAL CANNULAE AND USE THEREOF

(75) Inventors: Michael J. Valenti, Rochester, NY (US); Stephen P. Bartels, Pittsford, NY (US); Stephen R. Davio, Fairport, NY (US); Francesco P. Rotoli, Rochester, NY (US); Brian Levy, New York, NY (US)

Correspondence Address: Bausch & Lomb Incorporated, One Bausch & Lomb Place, Rochester, NY, 14604-2701, US

(73) Assignee: BAUSCH & LOMB INCORPORATED, Rochester, NY (US)

(21) Appl. No.: 12/812,171

(22) PCT Filed: Jan. 9, 2009
(86) PCT No.: PCT/US2009/030532

§ 371 Date: Aug. 9, 2010

Related U.S. Application Data
(60) Provisional application No. 61/020,162, filed on Jan. 10, 2008.

(51) Int. Cl. A61M 5/32

(52) U.S. Cl. 604/239

(57) ABSTRACT

An ophthalmic injection system having coaxial cannulae includes a plurality of syringe bodies and a plurality of cannulae, wherein a first non-flexible cannula is configured to accommodate and guide a second cannula that moves inside the first cannula and penetrates into the internal cavity of an eye globe. The first cannula is long enough to penetrate one or more outer layers of the eye globe. A syringe body is connected to the second cannula for accommodation and infusion of a pharmaceutical composition. A plunger is inserted into an annular space of the second syringe body at its distal end for injecting the pharmaceutical composition to the surgical site.

This application is a national-stage application, filed under 35 U.S.C. §371, of International Application PCT/US2009/030532, filed on Jan. 9, 2009, and claims the right of priority based on said International Application, which claims the right of priority based on U.S. Provisional Patent Application No. 61/020,162, filed on Jan. 10, 2008. Both International Application PCT/US2009/030532 and U.S. Provisional Patent Application No. 61/020,162 are incorporated herein by reference.

### FIELD

The present invention relates generally to an ophthalmic injection system and a method for administering a pharmaceutical composition using the injection system. More specifically, it relates to an ophthalmic injection system containing coaxial cannulae for intravitreal injection and a method for administering a pharmaceutical composition intravitreally using the injection system.

### BACKGROUND

Intravitreal injection is a method of delivering a therapeutic agent directly to an interior portion of the eye by using a needle passing through the pars plana. It has been reported that intravitreal injection of a therapeutic agent is effective to improve ophthalmic disease states such as diabetic retinopathy, macular degeneration, retinal venous occlusion, and retinal detachment. Thus, various therapeutic agents have been clinically tested for such conditions with intravitreal administration.

An intravitreal injection procedure, however, entails many potential risks, which include injury to ophthalmic tissues, infection, and bleeding. Further, researchers have found that the intravitreal bolus of a therapeutic agent tends to not remain at the intended injection site. This tendency of the bolus not to remain in place not only decreases maximal efficacy of the therapeutic agent but also may cause unexpected side effects. More specifically, upon intravitreal injection, part of the bolus may adhere to the needle and eventually be taken away with the needle upon withdrawal. This could result in deposition of some of the therapeutic agent near the vitreous base and cilliary body. It is generally undesirable to deposit a therapeutic agent intended to treat the retina or choroid away from the posterior segment.

It is believed that the matrix structure of the vitreous is partly or wholly attributable to the cause of the bolus behavior during injection. The vitreous is a solid gel with a network of collagen fibers. Intravitreal insertion of the needle displaces the vitreous and the surrounding vitreous structure moves in a fashion as to create a cavity where the needle is placed. The injection of a pharmaceutical composition increases the intraocular pressure which in turn, forces the injected materials up the needle track. As the needle is withdrawn, the tendency of the vitreous is to return to the pre-injection state and to push the bolus back toward the injection site. The force could stir the bolus away from the original injection site. Use of a small needle can reduce the undesirable effects but a small needle cannot penetrate the dense outer layers of the patient's eye. Therefore, there is a need for a device or method to administer a therapeutic agent that assures accurate drug delivery without causing the therapeutic agent to reflux or adhere to the needle.

Intravitreal injection has also been associated with an increase in exogenous endophthalmitis which results from direct inoculation as a complication of ocular injections. Organisms that reside at the conjunctiva, eyelid, or eyelashes and are introduced at the time of the injection usually cause post injection endophthalmitis.

Therefore, there is a continued need to provide novel or improved devices and methods for effecting intravitreal injection that minimizes severe trauma to an eye and avoids risks of post-surgical infection.

### SUMMARY OF THE INVENTION

One embodiment provides an ophthalmic injection system having a plurality of syringes comprising a first syringe body configured to accommodate a second syringe body moving along the inner contour of the first syringe body; a first non-flexible cannula formed at or attached to the distal end of the first syringe body to provide a guidance to a second cannula into the internal cavity (the interior) of the eye globe, or the posterior segment of a patient's eye, wherein the first cannula is sufficiently long to penetrate one or more outer layers of the patient's eye and has an inner diameter to properly guide a second cannula; the second syringe body for accommodation and infusion of a pharmaceutical composition, the second syringe body being inserted into the inner space of the first syringe body; the second cannula formed at or attached to the distal end of the second syringe body to deliver the pharmaceutical composition to a surgical site of the patient's eye, wherein the second cannula is inserted into the inner space of the first cannula and moves along the inner contour of the first cannula; and a plunger positioned in an annular space of the second syringe body for injecting the pharmaceutical composition to the surgical site.

Another embodiment provides an ophthalmic injection system comprising a first syringe body configured to accommodate a cannula positioning assembly for positioning a second cannula; a first non-flexible cannula formed at or attached to the distal end of the first syringe body to provide a guidance to a second cannula into the internal cavity (the interior) of the eye globe, or the posterior segment of the patient's eye, wherein the first cannula is long enough to penetrate one or more outer layers of the patient's eye and has an inner diameter to properly guide the second cannula; a cannula positioning assembly for positioning the second cannula into the internal cavity of the eye globe, or the posterior segment of a patient's eye through the first cannula, wherein the cannula positioning assembly is controllable manually or automatically in a manner that the tip of the second cannula is accurately positioned to a surgical site by an operator or a machine; a second syringe body for accommodation and infusion of a pharmaceutical composition, the second syringe body being placed outside the first syringe; a second cannula attached to the cannula positioning assembly to deliver the pharmaceutical composition to the surgical site of the patient's eye, wherein the second cannula is inserted into the inner space of the first cannula and moves along the inner contour of the first cannula; a conduit connecting the distal end of the second syringe body and the proximal end of the second cannula to allow passage of the pharmaceutical composition to the tip of the second cannula; and a plunger positioned in an annular space of the second syringe body for injecting the pharmaceutical composition to the surgical site.

Yet another embodiment provides a method for delivering a pharmaceutical composition into the interior of a patient's eye globe or the posterior segment of a patient's eye comprising the steps of: inserting a second cannula formed at or attached to a second syringe body to the inner space of a first cannula, wherein the first cannula is formed at or attached to one end of a first syringe body, a combination of the first syringe body and the first cannula is configured to accommodate the second syringe body and the second cannula; inserting the first cannula to a pre-determined position of a patient's eye such that the first cannula penetrates one or more outer layers of the patient's eye, thereby making an incision to allow insertion of the second cannula into the interior of the eye globe or the posterior segment; positioning the tip of the second cannula to a predetermined surgical site; injecting a pharmaceutical composition to the surgical site; and removing the first and second cannulae from the patient's eye.

Another embodiment provides a method for delivering a pharmaceutical composition into the cavity of an eye globe of a patient or the posterior segment of a patient's eye, the method comprising the steps of: inserting a first cannula formed at or attached to a first syringe body to a pre-determined site of a patient's eye such that the first cannula penetrates one or more outer layers of the patient's eye, thereby making an incision to allow insertion of a second cannula into the cavity of an eye globe or the posterior segment, wherein the first syringe body is configured to accommodate a cannula positioning assembly for positioning the second cannula; positioning the second cannula to a predetermined surgical site by manipulating the cannula positioning assembly, wherein the second cannula is connected to the distal end of the second syringe body placed outside the first syringe body through a conduit which allows passage of the pharmaceutical composition to the tip of the second cannula; injecting the pharmaceutical composition to the predetermined surgical site; and removing the first and second cannulae from the patient's eye.

Another embodiment provides an ophthalmic injection system comprising a first syringe body configured to accommodate a cannula positioning assembly for positioning a second cannula; a first non-flexible cannula formed at or attached to the distal end of the first syringe body to provide a guidance to the second cannula into the cavity of an eye globe of a patient or the posterior segment of a patient's eye, wherein the first cannula is only long enough to penetrate one or more outer layers of the patient's eye and not long enough to penetrate all layers of the eye, and has an inner diameter to properly guide a second cannula through any remaining layers; the cannula positioning assembly for positioning a second cannula into the cavity of an eye globe or the posterior segment through the first cannula, wherein the cannula positioning assembly is controllable manually or automatically in a manner that the tip of the second cannula is accurately positioned to a surgical site by an operator or a machine; a second syringe body for accommodation and infusion of a pharmaceutical composition, the second syringe body being placed outside the first syringe body; the second cannula being attached to the cannula positioning assembly to deliver the pharmaceutical composition to the surgical site of the patient's eye, wherein the second cannula is inserted into the inner space of the first cannula and moves along the inner contour of the first cannula; and a plunger positioned in an annular space of the second syringe body for injecting the pharmaceutical composition to the surgical site.

### BRIEF DESCRIPTION OF DRAWINGS

FIGS. 1a and 1b are cross-sectional views of an embodiment in accordance with the present invention;

FIG. 2 is a cross-sectional view of another embodiment in accordance with the present invention;

FIG. 3 is a perspective view of yet another embodiment in accordance with the present invention;

FIGS. 4a and 4b are perspective views of portions of the embodiment of FIG. 3. and

FIGS. 5A AND 5B are cross-sectional views of another embodiment, in accordance with the present invention.

### DETAILED DESCRIPTION

The following description is merely exemplary in nature and is not intended to limit the present disclosure, application, or uses.

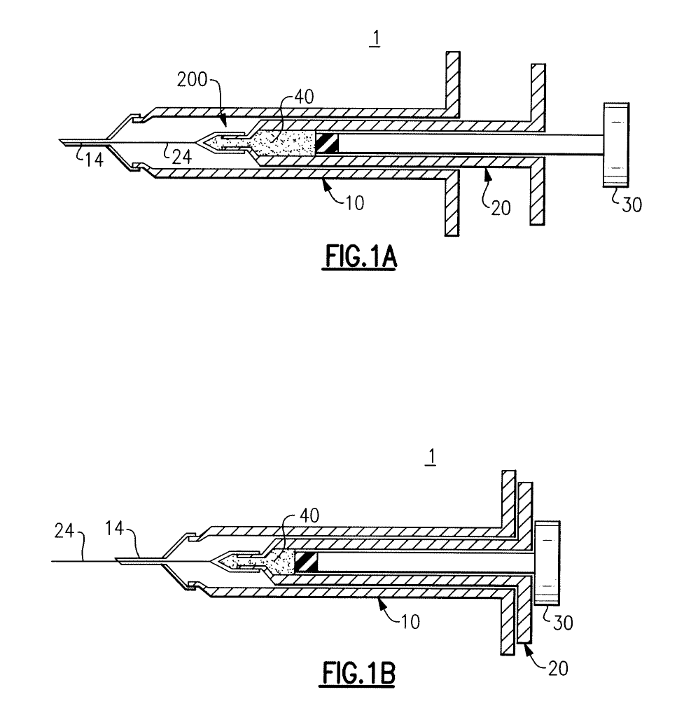

FIGS. 1a and 1b are illustrations of a first embodiment of an ophthalmic injection system 1, showing before and after intravitreal injection states respectively. Referring to FIG. 1a, ophthalmic injection system 1 comprises a first syringe body 10 configured to accommodate a second syringe body 20, and a first substantially non-flexible cannula 14 to provide guidance to a second cannula 24. The second syringe body 20 is for accommodation and infusion of a pharmaceutical composition 40. The second cannula 24 is preferably formed at or attached to a distal end 200 of the second syringe body 20 as shown, and a plunger 30 is positioned within second syringe body 20 for injecting the pharmaceutical composition 40 to the surgical site (such as, for example, an intravitreal surgical site) through cannula 24. The first and second cannulae 14 and 24 operate cooperatively to minimize undesirable effects of intravitreal injection, and the ophthalmic injection system 1 comprising these cannulae 14 and 24 is able to perform effective delivery of the pharmaceutical composition to the surgical site (such as, for example, an intravitreal surgical site). Therefore, the system 1 is useful in providing treatment of various ophthalmic conditions, especially those needing intravitreal injection for achievement of desirable results. Some of the advantages of the present invention can be further understood from the following discussion.

The first syringe body 10 is configured to accommodate the second syringe body 20 which is designed to move along the inner contour of the first syringe body 10. The configuration of the first syringe body 10 is not limited to traditional syringe shapes as long as the design allows the second syringe body 20 to move inside the first syringe body 10, for example in some embodiments, as illustrated herein. In one embodiment, the inner cross section of the first syringe body 10 and the outer cross section of the second syrineg body 20 have generally similar shapes. In another embodiment, the first and second syringe bodies 10 and 20 are concentric to achieve proper sliding movement of the second syringe body 20. In still another embodiment, the centers of the first and second syringe bodies 10 and 20 are offset, but the second syringe body 20 is still movable inside the first syringe body 10. The first syringe body 10 may have a distal tip configured to provide a connection site to which the first non-flexible cannula 14 may be attached. Alternatively, the first cannula 14 may be formed at the distal tip of the first syringe body 10 and the cannula 14 and the syringe body 10 may be manufactured as a single member. If the first cannula 14 is attachable to the distal tip, the first syringe body 10 may be designed as a reusable unit. The first syringe body 10 may be made of any materials suitable for its intended purpose, regardless of its reusability.

The first substantially non-flexible cannula 14 is formed at or attached to the distal end of the first syringe body 10 to provide guidance to the second cannula 24 into the interior of a patient's eye globe or the posterior segment of a patient's eye (not shown). The combination of first syringe body 10 and first cannula 14 works as one unit in operation. The main function of the first cannula 14 is to create an incision passing through one or more outer layers of the patient's eye. The outer layers of a human eye include, among others, the scleral wall which is dense connective tissue filled with the protein collagen. Such tough tissue is strong enough to prevent penetration of micrometer-sized cannulae, which, however, are advantageously used to reduce undesirable effects of intravitreal injection. Therefore, the incision created by the first cannula 14 assists easy penetration of the second cannula 24 having a relatively small diameter. For proper operation of the system, the first cannula 14 needs to be long enough to penetrate at least the scleral wall or all layers of the eye (e.g., wall layers) so that the second cannula 24 is not required to go through the dense layer or layers. Depending on the design criteria and the surgical procedure, the desired length of the first cannula 14 may vary. To create an incision, it is preferable for the first cannula 14 to be fabricated of a rigid or non-flexible material. Any conventional rigid or non-flexible material can be used so long as it is acceptable for an ophthalmic surgery. Preferably, the rigid or non-flexible material is metal but may be other materials, such as polymeric materials (e.g. polyimide) or other materials. The inner contour of the first cannula 14 serves as a guide for the second cannula 24 to move through the first cannula 14.

The second syringe body 20 accommodates a pharmaceutical composition 40 to be infused to a surgical site of the patient's eye through cannula 24. As shown in FIG. 1a, the second syringe body 20 is inserted into the inner space of the first syringe body 10 like a plunger prior to operation. Once an incision is created in an outer layer of the patient's eye by the first cannula 14, the second syringe body 20 is pushed forward to place the second cannula 24 at the surgical site to be treated with pharmaceutical compositions 40. After the operation, the second syringe body 20 is pulled away from the eye together with the first syringe body 10. The second syringe body 20 has a distal tip configured to provide a connection site to which the second cannula 24 can be attached or formed. The second syringe body 20 is made of any conventional materials used for syringes.

The second cannula 24 is formed at or attached to the distal end of the second syringe body 20 to deliver the pharmaceutical composition 40 to a surgical site of the patient's eye. The second cannula 24 is inserted into the inner space of the first cannula 14 prior to operation and is positioned to a surgical site through sliding movement within the first cannula 14. The second cannula 24 is sized so as to reduce undesirable effects caused by intravitreal injection such as reflux and adherence to the cannula. It is generally desirable to employ, as the second cannula 24, a cannula having a small outer diameter. In one aspect, the second cannula 24 of a device of the present invention may have an outer diameter no greater than about 1000 μm (micrometer). In another aspect, the second cannula 24 may also have an outer diameter of less than about 500 μm, or less than about 400 μm, or less than about 300 μm, or less than about 250 μm, or less than about 200 μm. In still a further aspect, the second cannula 24 may have an outer diameter of about 32 ga. or less. In one embodiment, the outer diameter is from about 500 μm to about 900 μm, or from about 200 μm to about 500 μm. The inner diameter of the second cannula 24 may not be critical in achieving the goal of the invention but needs to allow for sufficient flow of composition 40 to the surgical site. The second cannula 24 can be made of either a flexible or non-flexible material. Whether formed of a flexible or non-flexible material, cannula 24, because of its small outer diameter, cannot be inserted to an intravitreal site due to the scleral wall under normal circumstances; but an incision created by the first cannula 14 enables use of cannula 24 having a small outer diameter (in particular, a cannula having an outer diameter of about 300 μm or less). In one embodiment, the second cannula 24 is a metal cannula.

The plunger 30 is for injecting the pharmaceutical composition 40 to the surgical site and inserted into the inner space of the second syringe body 20. The plunger 30 can be any conventional plunger suitable for use in an ophthalmic treatment or surgery.

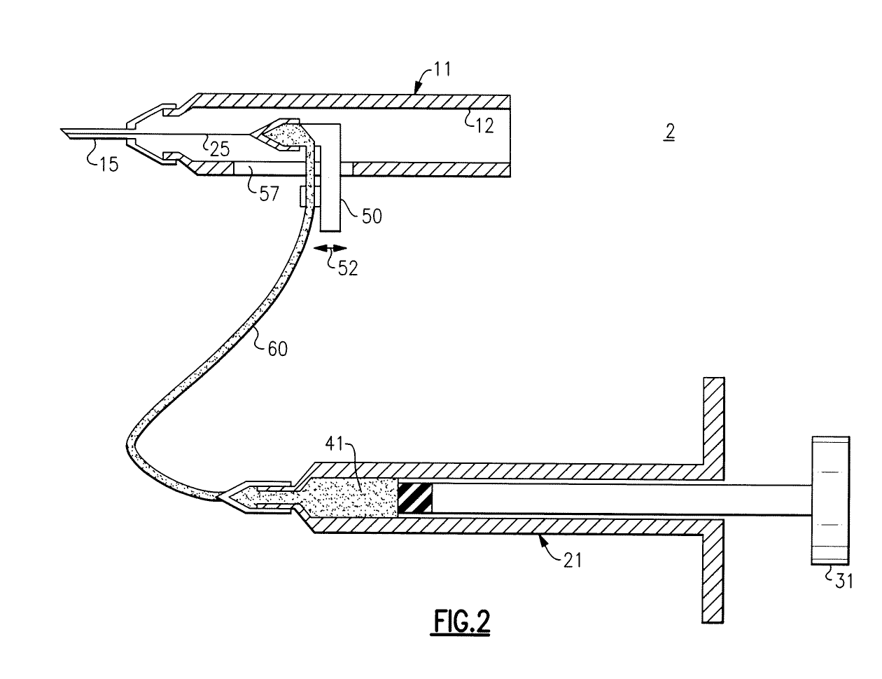

FIG. 2 is an illustration of another embodiment of the present invention, in which a system 2 contains a cannula positioning assembly 50 to guide and adjust the position of a second cannula 25, and a second syringe body 21 apart from a first syringe body 11. Referring to FIG. 2, the ophthalmic injection system 2 comprises the first syringe body 11 configured to accommodate the cannula positioning assembly 50 for positioning the second cannula 25, a first non-flexible cannula 15 formed at or attached to the distal end of the first syringe body 11, the cannula positioning assembly 50 for positioning the second cannula 25 into the cavity of the eye globe of a patient or the posterior segment of a patient's eye through the first cannula, the second syringe body 21 for accommodation and infusion of a pharmaceutical composition 41, a conduit 60 connecting the distal end of the second syringe body 21 and the proximal end of the second cannula 25, and a plunger 31 for injecting the pharmaceutical composition 41 to the surgical site (not shown).

The first syringe body 11 is configured to accommodate a cannula positioning assembly 50 for positioning a second cannula 25 and the first non-flexible cannula 15 is formed at or attached to the distal end of the first syringe body 11.

The cannula positioning assembly 50 for positioning a second cannula 25 carries and positions the second cannula 25 into the cavity of the eye globe of a patient or the posterior segment of a patient's eye through the first cannula 15. The cannula positioning assembly 50 is attached to the second cannula 25 and mounted in the first syringe body 11, such that the cannula positioning assembly 50 can adjust the position of the tip of the second cannula 25 relying on guidance of the first cannula 15. The configuration of the cannula positioning assembly 50 may be any structure able to allow movement of the second cannula 25 within the first syringe body 11. In the embodiment shown, the first syringe body 11 has a longitudinal slot 51 in which a portion of the cannula positioning assembly 50 is mounted so as to allow its movement along the slot 51 of the first syringe body 11, as indicated by arrows 52. The cannula positioning assembly 50 may be configured as shown, to have a handle for an operator's positioning of the second cannula 25. The cannula positioning assembly 50 is controllable manually or automatically so that the tip of the second cannula 25 is accurately positioned at a surgical site by an operator or a machine. The cannula positioning assembly 50 may be one of many other configurations than the slider arm assembly 50 shown. As those skilled in the art will understand, a cannula positioning assembly 50 may be any configuration capable of moving cannula 25 from a retracted position to an extended position relative to cannula 15.

In the embodiment illustrated by FIG. 2, the second syringe body 21 is placed outside the first syringe 11 and the second cannula 25 is attached to the cannula positioning assembly 50. Therefore, the system requires a conduit 60 connecting the distal end of the second syringe body 21 and the proximal end of the second cannula 25. The conduit 60 allows passage of the pharmaceutical composition 41 to the tip of the second cannula 25. The conduit 60 can be made of any conventional material. In one aspect, conduit 60 is made of a flexible material.

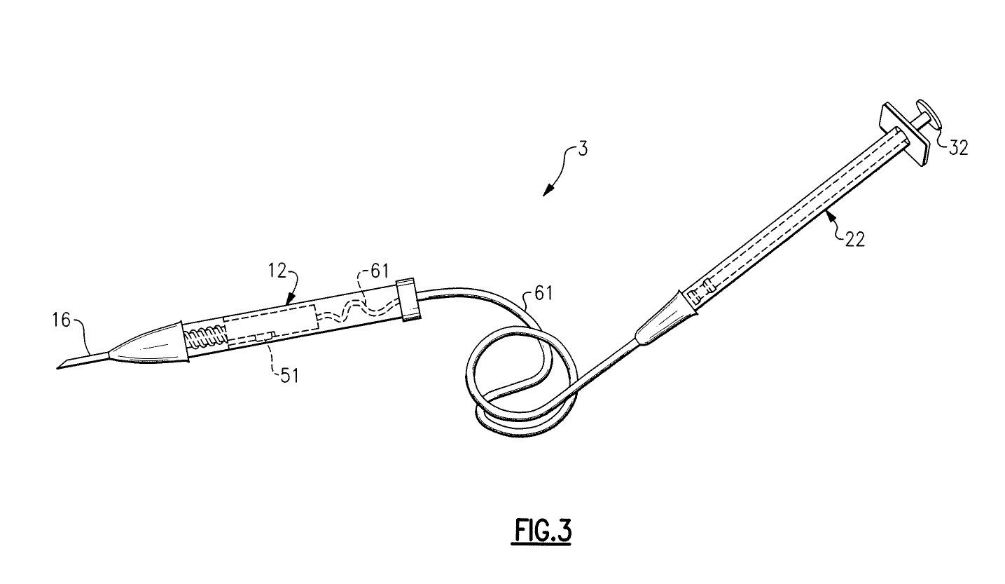

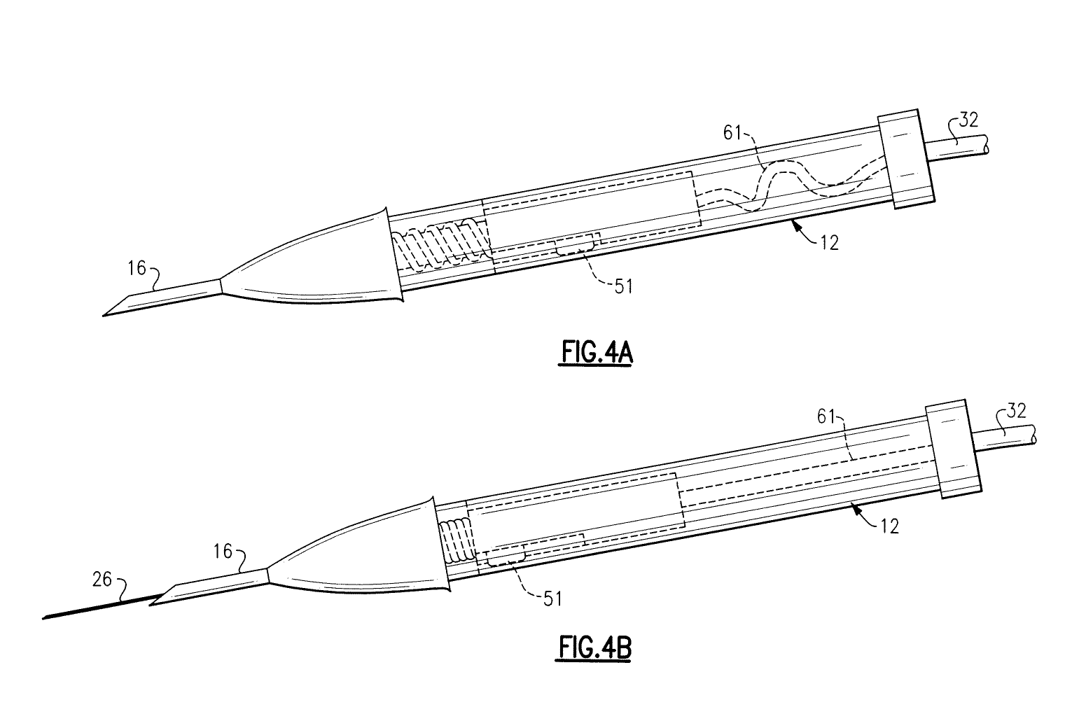

A variation of the embodiment of FIG. 2 is illustrated with FIGS. 3, 4a, and 4b. While the conduit connections are different, the other features of this embodiment are generally identical to those of FIG. 2. FIG. 3 shows the entire ophthalmic injection system 3 and FIGS. 4a and 4b illustrate manipulation of a first syringe unit comprising a first syringe body 12, a first cannula 16, a second cannula 26 and a cannula positioning assembly 53. In FIG. 4a, the cannula positioning assembly 53 is placed in a retracted position where the second cannula 26 is positioned not to protrude beyond the first cannula 16. Upon insertion of the first cannula 16 into the patient's eye, the cannula positioning assembly 53 is pushed forward, as shown in FIG. 4b, to place the tip of the second cannula 26 at a surgical site (not shown). The syringe body 22, plunger 32, and conduit 61 are essentially the same as the corresponding structure of syringe body 21, plunger 31, and conduit 60, as described in FIG. 2. The conduit 61 attaches to assembly 53, internally of body 12, as shown by the dashed lines.

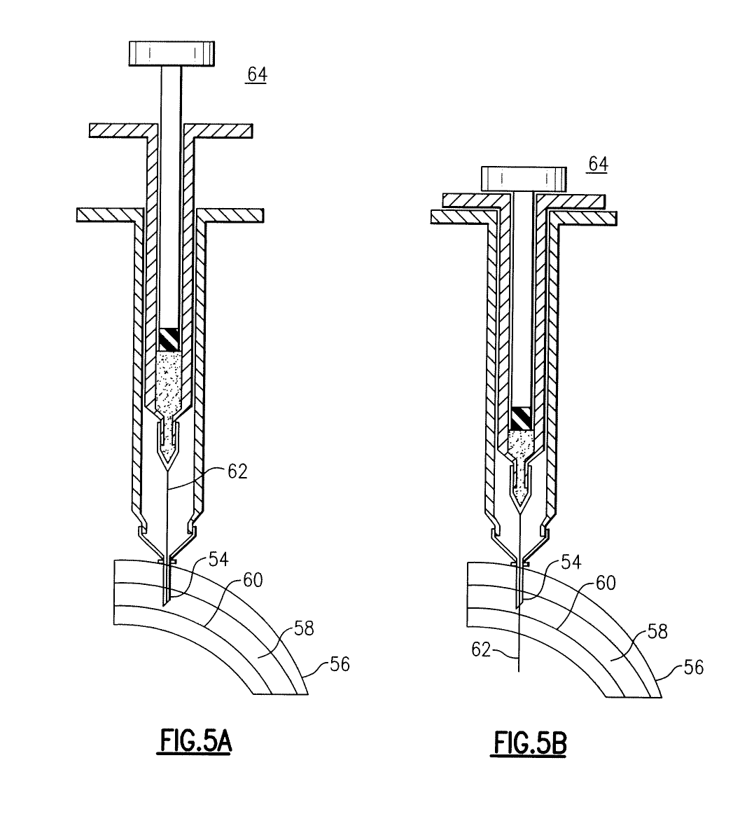

FIGS. 5a and 5b show another embodiment of the present invention. FIGS. 5a and 5b are essentially the same as the embodiment described above at FIGS. 1a and 1b. The difference is that first cannula 54 is only long enough to penetrate one or more out layers of the patient's eye and not long enough to penetrate all layers of the eye. This is shown in FIG. 5a, where cannula 54 has penetrated conjunctiva 56 and partially penetrated sclera 58. Cannula 54 has not fully penetrated sclera 58 and has not penetrated cilliary body 60 at all. Cannula 54 preferably has an inner diameter to properly guide a second cannula 62 through any remaining layers (as shown in FIG. 5b). In all other respects the injection system 64 is the same as that described at FIGS. 1a and 1b.

System 64 can help reduce the risk of infection. By not allowing cannula 54 to penetrate into the cavity of the eye globe or the posterior segment, organisms that reside at the conjunctiva, eyelid, or eye lashes are much less likely to be introduced into the interior or internal cavity of the eye globe or the posterior segment when cannula 62 is inserted through the remaining layers of the eye. It is believed that this will decrease exogenous endophthalmitis resulting from direct inoculation of the eye.

The devices described herein and illustrated in FIG. 1 a-4b, provide for a method of delivering a pharmaceutical composition into the posterior segment of a patient's eye. The method comprises the steps of inserting a second cannula into the inner space of the first cannula, inserting a first cannula at a pre-determined position of a patient's eye to create an incision, positioning the tip of the second cannula at a predetermined surgical site, injecting the pharmaceutical composition, and removing the first and second cannulae from the patient's eye.

The method begins with preparing the system to be insertable to the patient's eye. The second cannula and second syringe body unit are partially inserted to the inner space of the first cannula, such that the tip of the second cannula does not protrude from the first cannula. The first syringe body and first cannula unit in this step should be configured to accommodate and guide the second syringe body and second cannula unit to be used. The first cannula surrounding the second cannula is inserted at a pre-determined position of the patient's eye, such that the first cannula penetrates the outer layers of the patient's eye partially or entirely. This step is necessary to make an incision through the relatively dense outer layers of the eye. The incision is to enable placement of the second cannula into the interior of the eye globe or the posterior segment. Thus, the first cannula, at minimum, has to pass through the dense layer or layers of the eye, and therefore, needs to be sufficiently robust and rigid for this purpose. Thereafter, the tip of the second cannula is positioned to a predetermined surgical site inside the cavity of the eye globe or the posterior segment through the incision and injection of a pharmaceutical composition follows. After completion of the injection, the first and second cannulae are removed from the patient's eye.

An intravitreal injection of the present invention can be applied to effect injection of a variety of pharmaceutical compositions. The pharmaceutical composition typically contains at least one active agent to treat ophthalmic disease states. Examples of an active agent of the composition includes, but are not limited to, an antibiotic agent, a beta blocker, a corticosteroid agent, an anti-inflammatory agent, an adrenergic receptor agonist or antagonist, a VEGF inhibitor, an enzymatic agent, a neuroprotective agent, an anti-cancer agent, anti-fibrotic agent, anti-proliferative agent, tumor necrosis factor-α (“TNF-α”) inhibitors, and an ophthalmically acceptable therapeutic agent. Preferably, the active agent can be an enzymatic agent or a precursor thereof (such as plasmin, plasminogen and tissue plasminogen activator), a VEGF inhibitor such as pegaptanib, bevacizumab, and ranibizumab, a corticosteroid agent, such as triamcinolone, TNF-α inhibitors, erythropoietin, or a mixture thereof.

The versatile applicability enables an intravitreal injection method of the invention to be applied to treatment of a variety of ophthalmic diseases. An intravitreal injection method of the invention, therefore, can be used to treat any ophthalmic disease requiring intravitreal injection. Preferably, a method of the invention can be applied to treat retinopathy such as hypertensive retinopathy, diabetic retinopathy and solar retinopathy; macular degeneration such as age-related macular degeneration; retinal venous occlusion such as nonischemic retinal venous occlusion or ischemic retinal venous occlusion; macular edema; or retinal detachment such as rhegmatogenous retinal detachment, exudative retinal detachment, or tractional retinal detachment.

The devices described herein also provide a method for delivering a pharmaceutical composition into the posterior segment of a patient's eye comprising the steps of: inserting a first cannula formed at or attached to a first syringe body at a pre-determined position of a patient's eye such that the first cannula penetrates one or more outer layers of the patient's eye, thereby making an incision to allow insertion of a second cannula into the interior of the eye globe or the posterior segment, wherein the first syringe body is configured to accommodate a cannula positioning assembly for positioning a second cannula; positioning the second cannula to a predetermined surgical site by manipulating the cannula positioning assembly; injecting a pharmaceutical composition to the surgical site; and removing the first and second cannulae from the patient's eye. In one embodiment, the second cannula is connected to the distal end of the second syringe body placed outside the first syringe body through a conduit which allows passage of the pharmaceutical composition to the tip of the second cannula. In another embodiment, the second syringe body is placed inside the first syringe body.

As various modifications could be made in the constructions and methods herein described and illustrated without departing from the scope of the invention, it is intended that all matter contained in the foregoing description or shown in the accompanying drawings shall be interpreted as illustrative rather than limiting. Thus, the breadth and scope of the present invention should not be limited by any of the above-described exemplary embodiments, but should be defined only in accordance with the following claims and their equivalents.

What is claimed is:

1. An ophthalmic injection system having coaxial cannulae comprising:
a first syringe body configured to accommodate a second syringe body moving along an inner contour of the first syringe body;
a first substantially non-flexible cannula formed at or attached to a distal end of the first syringe body to provide guidance to a second cannula into an interior of a patient's eye globe or a posterior segment of a patient's eye, wherein the first cannula is sufficiently long to penetrate one or more outer layers of the patient's eye and has an inner diameter to properly guide the second cannula;
wherein the second syringe body accommodates a pharmaceutical composition for infusion thereof into the interior of a patient's eye globe or the posterior segment of a patient's eye;
wherein the second cannula is formed at or attached to a distal end of the second syringe body to deliver the pharmaceutical composition to a surgical site of the patient's eye, wherein the second cannula is inserted into an annular space of the first cannula and moves along the inner contour of the first cannula; and
a plunger contained within the second syringe body for injecting the pharmaceutical composition to the surgical site.

2. The ophthalmic injection system of claim 1, wherein the second cannula has an outer diameter less than about 1000 micrometers.

3. The ophthalmic injection system of claim 1, wherein the second cannula has an outer diameter less than about 500 micrometers.

4. The ophthalmic injection system of claim 1, wherein the second cannula has an outer diameter less than about 250 micrometers.

5. The ophthalmic injection system of claim 1, wherein the first cannula is only sufficiently long to penetrate one or more outer layers of the patient's eye and not sufficiently long to penetrate all layers of the eye.

6. An ophthalmic injection system having coaxial cannulae comprising:
a first syringe body configured to accommodate a cannula positioning assembly;
a first substantially non-flexible cannula formed at or attached to a distal end of the first syringe body to provide guidance to a second cannula into an interior of a patient's eye globe or a posterior segment of a patient's eye, wherein the first cannula is long enough to penetrate one or more outer layers of the patient's eye and the first cannula has an inner diameter to properly guide the second cannula;
the cannula positioning assembly for positioning the second cannula into the interior of a patient's eye globe or the posterior segment of a patient's eye through the first cannula;
a second syringe body for accommodation and infusion of a pharmaceutical composition, the second syringe body being placed outside the first syringe body;
wherein a conduit connects the second syringe body to the second cannula to allow passage of the pharmaceutical composition to the tip of the second cannula; and
a plunger contained within the second syringe body for injecting the pharmaceutical composition through the conduit and the second cannula to a surgical site.

7. The ophthalmic injection system of claim 6, wherein the second cannula has an outer diameter less than about 1000 micrometers.

8. The ophthalmic injection system of claim 6, wherein the second cannula has an outer diameter less than about 500 micrometers.

9. The ophthalmic injection system of claim 6, wherein the second cannula has an outer diameter less than about 500 micrometers.

10. The ophthalmic injection system of claim 6, wherein the first cannula is only long enough to penetrate one or more outer layers of the patient's eye and not long enough to penetrate all layers of the eye.

11. A method for delivery of a pharmaceutical composition into a patient's eye comprising the steps of:
providing a first syringe body, which has a first cannula formed at or attached to one end of the first cannula;
further providing a second syringe body, which has a second cannula formed at or attached to one end of the second syringe body, wherein the second syringe body and second cannula are contained within the first syringe body and first cannula;
inserting the first cannula into the patient's eye, such that the first cannula penetrates one or more outer layers of the patient's eye;
inserting the second cannula through the first cannula into the patient's eye towards a surgical site;
injecting the pharmaceutical composition, which is contained in the second syringe body, to a surgical site; and
removing the first and second cannulae from the patient's eye.

12. The method for delivering a pharmaceutical composition of claim 11, wherein the second cannula has an outer diameter less than about 1000 micrometers.

13. The method for delivering a pharmaceutical composition of claim 11, wherein the second cannula has an outer diameter less than about 300 micrometers.

14. The method for delivering a pharmaceutical composition of claim 11, wherein the first cannula is only long enough to penetrate one or more outer layers of the patient's eye and not long enough to penetrate all layers of the eye.

15. The method for delivering a pharmaceutical composition of claim 11, wherein the pharmaceutical composition comprises at least one active agent to treat ophthalmic diseases.

16. The method for delivering a pharmaceutical composition of claim 15, wherein the active agent is plasmin, plasminogen, tissue plasminogen activator, pegaptanib, bevacizumab, ranibizumab, triamcinolone, a TNF-α inhibitor, erythropoietin, or mixture thereof.

17. The method for delivering a pharmaceutical composition of claim 11, wherein the method is useful to treat diabetic retinopathy, macular degeneration, retinal venous occlusion, vitreal macular traction, macular pucker, macular hole, macular edema, or retinal detachment.

18. A method for delivering a pharmaceutical composition into an interior of a patient's eye globe or a posterior segment of a patient's eye, the method comprising the steps of:
inserting a first cannula formed at or attached to a first syringe body to a pre-determined position of the patient's eye such that the first cannula penetrates one or more outer layers of the patient's eye, thereby making an incision to allow insertion of a second cannula into the interior of the patient's eye globe or the posterior segment of the patient's eye, wherein the first syringe body is configured to accommodate a cannula positioning assembly for positioning the second cannula;
positioning the second cannula at a predetermined surgical site by manipulating the cannula positioning assembly, wherein the second cannula is connected to a second syringe body apart from the first syringe body through a conduit;
injecting the pharmaceutical composition from the second syringe body through the conduit and second cannula to a surgical site; and
removing the first and second cannulae from the patient's eye.

19. The method for delivering a pharmaceutical composition of claim 18, wherein the second cannula has an outer diameter less than about 1000 micrometers.

20. The method for delivering a pharmaceutical composition of claim 18, wherein the second cannula has an outer diameter less than about 300 micrometers.

21. The method for delivering a pharmaceutical composition of claim 18, wherein the first cannula is only long enough to penetrate one or more outer layers of the patient's eye and not long enough to penetrate all layers of the eye.

22. The method for delivering a pharmaceutical composition of claim 18, wherein the pharmaceutical composition comprises at least one active agent to treat ophthalmic diseases.

23. The method for delivering a pharmaceutical composition of claim 22, wherein the active agent is plasmin, plasminogen, tissue plasminogen activator, pegaptanib, bevacizumab, ranibizumab, triamcinolone, a TNF-a inhibitor, erythropoietin, or mixture thereof.

24. The method for delivering a pharmaceutical composition of claim 18, wherein the method is useful to treat diabetic retinopathy, macular degeneration, retinal venous occlusion, macular edema, or retinal detachment.
